SEQUENCE LISTING (8 sequences)

SEQ ID NO: 1 - DNA, 21 bp, Artificial Sequence
  Description of Artificial Sequence Synthetic oligonucleotide
ccuaaacgaugcuauauuutt

SEQ ID NO: 2 - DNA, 21 bp, Artificial Sequence
  Description of Artificial Sequence Synthetic oligonucleotide
aaauauagcaucguuuaggtt

SEQ ID NO: 3 - DNA, 21 bp, Artificial Sequence
  Description of Artificial Sequence Synthetic oligonucleotide
gccuuauuaaugauuaucutt

SEQ ID NO: 4 - DNA, 21 bp, Artificial Sequence
  Description of Artificial Sequence Synthetic oligonucleotide
agauaaucauuaauaaggctt

SEQ ID NO: 5 - DNA, 21 bp, Artificial Sequence
  Description of Artificial Sequence Synthetic oligonucleotide
guauagaugguauagguautt

SEQ ID NO: 6 - DNA, 21 bp, Artificial Sequence
  Description of Artificial Sequence Synthetic oligonucleotide
auaccuauaccaucuauactt

SEQ ID NO: 7 - DNA, 2941 bp, Homo sapiens
cctgtggccgctcccggctcggagtgtgatctaagcaggttgcgtaccttcctcaggtgactccggccacagcccattgtccgcggccaccggcggagtttagccgcagacctcgaagcgccccggggtccttcccgaacggcagcggctgcggcgggtccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagcttcattcagtgtgtgtgcgttggttgccgatcgtactgccagtagcattgtctgtctgtccggtcttgtttgtacattccattttcaattgttacagatgtgaactttattccttgtcactaattatatttaaaattatttctaggaagtcaaataaatataataaagggttgagccctctactttcttcttgccacctttttgtggcaatattaaagtgaactgctaatagtgtaagtacgtgcacaaaaccactgccagataaccagaggggcctgggaagggagaagaattagtgtatttttttcaaatagtacagtaatttgcctcataagcataggagcattgggaatgagagggaactgtgcccagtatactgttttttttcttcctccaataaaagtggtgtagtgccgaaagtgctaaaatatttagtgcggtattgctctgtgaattcaagttcaacagacttcactttggtcatgtttattaaaccaccagtgacatttaaaaatatatttttagcagtcgtaatgttagtcaccaagggaaggtggtggaatgtctatgtttttgattttactgtgagttaaaaaggcacatttctaccttctattgtttttaaattcaagaatagggaattagttcctggtgttgtttacgagtgtattctcgtgtcaacatacagggatttagacatttaactctctgtgccttgataagaatatcatttagagtgtagatacttttgcctttttaaaaaagccattattttatgagacttagtactcacactgcaaataactagtcagctcagttttaactttataggtttattgagtttcctttgtgtgatccatgtagatgcctcaaaatgtttcttcttcttcttttttttaatcttataagatatttttctaagtatttccagaaacatttgagagtgcccatcattttcaggtctgcagaaccatagcttccacgcacctgaacgagcacagaatgaactgacggtggaagacattatgagctgtgtccaacgttttaaccaaagcgtatcgtaccaacgatctgtgaaaatgcactggaagcttctggtcccggtttcctttgtggtctatgtgggtcttgtcctcattgtaactccgtatagatggtataggtattttaatcctggaagctgttgccttattaatgattatcttaaaatttcctccattggggcagcgtgggccaaattaaaacaaacaaaaccgcaactcctccacagaaacacaaacacagttattccatgaagtttagtatttggttgacatagtgctcttcaaattcatcccattaccctaaaagtaataactttgatgcttgctttaactttagtcccatctctgccactttgatgctatttgggttatgatggggcaagatggcagaggtattgggtttttttgtttttttccattcctctctacttctgtttcctagctttttctttctggagtttaagtacagtgatggttggcttgagtacctttttaaatctagcccagtataaacattagcctgcttaatatttagacatttataggtagaattctgagcactcaactcatgtttggcattttaaagtaaaaacaagtgtgacttcgaggaccaaagaaattgtcagctatacatttatctttatgaactcatttatattcctttttaatgactcgttgttctaacatttcctagaagtgttcttataaaggtctaatgtatccacaggctgttgtcttattagtaaatgcaaagtaatgactttgtctgttttactctagtctttagtacttcaaaattaccttttcatatccatgatcttgagtccatttgggggatttttaagaatttgatgtatttcaatacactgttcaaaattaaattgtttaattttatgtatgagtatgtatgttcctgaagttggtcctatttaaattattaaactattgtaactttgttcttgtgaaaaaaaaaaaaaaaaaa

SEQ ID NO: 8 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

(19) United States
(12) Patent Application Publication
(10) Pub. No.: US 2010/0297015 A1
(43) Pub. Date: Nov. 25, 2010

(54) CANCER BIOMARKER AND USES THEREOF

(76) Inventor: Shijie CAI, Oxford (GB)

Correspondence Address: Jeffrey J. King, Esq.;Patent Networks Law Group PLLC, 5000 Carillon Point, Suite 400, Kirkland, WA, 98033, US

(21) Appl. No.: 12/762,305

(22) Filed: Apr. 17, 2010

(30) Foreign Application Priority Data
Oct. 17, 2007 (GB) GB0720334.2
Oct. 16, 2008 (GB) PCT/GB2008/003497

(51) Int. Cl. A61K 49/00; C12Q 1/34; C12Q 1/68; A61K 31/7105; A61K 31/505; A61P 35/00

(52) U.S. Cl. 424/9.1; 435/18; 435/6; 514/44 A; 514/272

(57) ABSTRACT

A method of determining the cancer status of a subject, comprising the steps of (a) providing a sample of material obtained from a subject; (b) determining the level GTPCH in the sample; and (c) comparing the level determined in (b) with one or more reference values.

The present invention relates to a novel biomarker for cancer, and to uses of the novel biomarker

Despite the enormous advances in the understanding of cancer biology, about two-thirds of all cancer patients are still diagnosed at a relatively late stage. An American Cancer Society's report predicted there would be at least half a million deaths from an estimated over one million new cancer cases in the USA in 2006. Such poor prognosis in cancer patients is due to the complexity of the disease process by which cancer cells grow excessively, invade surrounding tissue continuously and metastasise to other organs.

During disease progression, cancer cells produce so-called biomarkers, which can be measured in samples from a patient, such as a patient's blood, urine, cerebrospinal fluid or tissue. The identification of cancer biomarkers suitable for early detection and diagnosis of cancer holds great promise for improving the clinical outcome of patients (Hartwell, L. et al (2006) Nat Biotechnol 24(8), 905-908).

A handful of cancer biomarkers are currently used routinely for population screening, disease diagnosis, prognosis, monitoring of therapy, and prediction of therapeutic response. Unfortunately, most of these biomarkers suffer from low sensitivity, specificity, and predictive value, particularly when applied to rare diseases in population screening programs. There is therefore a need for new cancer biomarkers that will further enhance the ability to diagnose cancer. In particular, there remains a need for a simple, rapid, reliable, reproducible method for diagnosing cancer.

According to a first aspect, the invention provides a method of determining the cancer status of a subject, comprising the steps of:

(a) providing a sample of material obtained from a subject;
(b) determining the level GTP cyclohydrolase in the sample; and
(c) comparing the level determined in (b) with one or more reference values.

GTP cyclohydrolase (GTPCH) is a novel biomarker for cancer, and is referred to herein as “the biomarker”.

GTPCH is encoded by the GCH gene (EC 3.5.4.16) on human chromosome 14q21.1-22.2. Mutations of this gene cause Segawa's disease, or hereditary progressive dystonia or dopa-responsive dystonia (DRD). This autosomal dominant disorder is characterized by childhood-onset dystonia with diurnal fluctuation (Blau N et al, Mol Genet Metab 2001; 74(1-2):172-85). Three different GCH cDNA isoforms have been identified in human liver, which are identical in their 5′ regions but differ at their 3′ ends (Togari A et al, Biochem Biophys Res Commun 1992; 187:359-65). Only the full length type I GTPCH generates the active enzyme (Giltlich M et al, The Biochemical journal 1994; 302:215-21; Cai S et al, Cardiovasc Res 2002; 55:838-49).

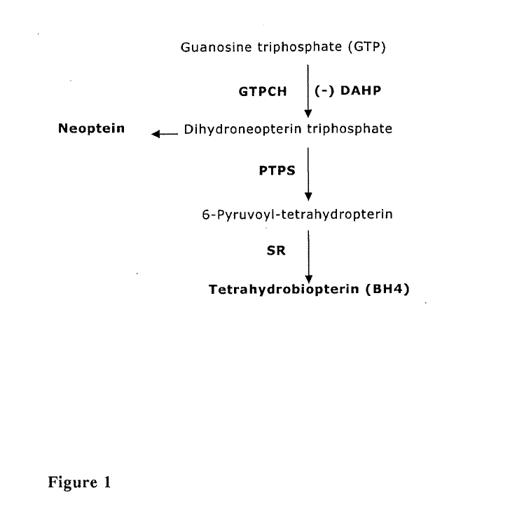

Type I GTPCH is the rate-limiting enzyme for synthesis of neopterin and tetrahydrobiopterin (BH4); BH4, in particular, is an essential cofactor for the activity of aromatic acid hydroxylases and all nitric oxide synthases (NOS) (Thony B et al, The Biochemical journal 2000; 347 Pt 1:1-16; Cai S et al, Cardiovasc Res 2005; 65(4):823-31) (FIG. 1). Activity of GTPCH can be inhibited by the low molecular weight inhibitor, 2,4-diamino-6-hydroxypyrimidine (DAHP)

The method of the invention may be used in conjunction with an assessment of clinical symptoms.

The phrase “cancer status” includes any distinguishable manifestation of cancer. For example, cancer status includes, without limitation, the presence or absence of cancer, the risk of developing cancer, the stage of the cancer, the progression of the cancer (e.g. progress of cancer or remission of cancer over time) and the effectiveness or response of a subject to treatment for a cancer.

The method of the invention may be used, for example, for any one or more of the following: to diagnose cancer in a subject; to assess the chance of a subject developing cancer; to advise on the prognosis for a subject with cancer; to monitor disease progression; and to monitor effectiveness or response of a subject to a treatment.

Preferably the method allows the diagnosis of cancer in a subject from the analysis of the level of the biomarker in a sample provided by the subject.

The cancer may be a cancer of a tissue selected from the group comprising lung, breast, brain, breast, pancreas, neck, prostate, bone, ovary, colon, liver, testis, endometrium, muscle, fat tissue, fibrous tissue, soft tissue, squamous tissue, skin tissue and lymphoma tissue, or combinations thereof.

The sample material obtained from the subject may comprise whole blood, blood serum, blood plasma, urine, mucous, fat tissue (adipose), lung tissue, brain tissue, pancreatic tissue, neck tissue, testis tissue, endometrium tissue, liver tissue, bone tissue, gastro-intestinal tissue, colon tissue, breast tissue, prostate tissue, lymphoma tissue, muscle tissue, fibrous tissue, soft tissue, squamous tissue, skin tissue, ovary tissue, fat tissue, lymphoma tissue or any other tissue or bodily fluid. Preferably, the sample is a sample of tissue.

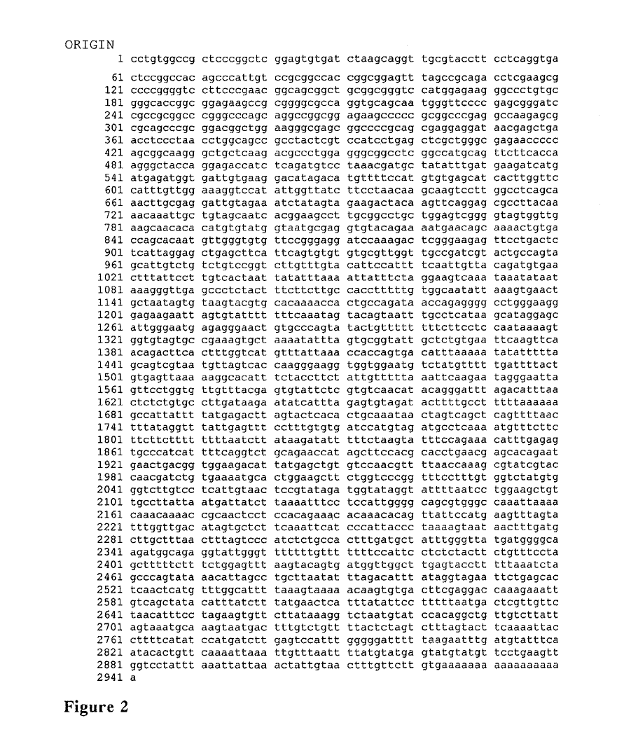

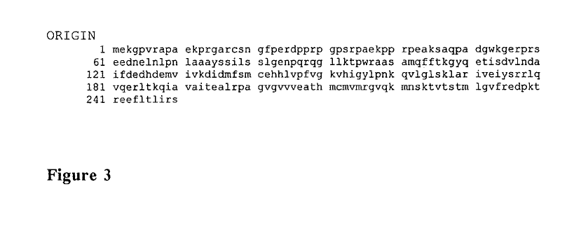

The mRNA sequence of the active form of human GTPCH can be found in the GenBank database and is accessible via accession number NM-000161. The mRNA sequence is given in FIG. 2. The corresponding protein has the accession number NP-000152. The protein sequence is given in FIG. 3.

Preferably, determination of the level of GTPCH in a sample comprises the detection of a polypeptide or an mRNA with at least 65% sequence identity, more preferably at least 70%, 75%, 80%, 85%, 90% or 95% sequence homology, to the sequence of GTPCH in FIG. 2 or 3.

Whilst the GTPCH protein exists naturally as a decamer, the decameric or the monomeric form may be detected in the method of the invention. Preferably the monomeric form is detected in the method of the invention.

Proteins frequently exist in the body, and in samples derived there from, in a plurality of different forms. These forms can result from either or both of pre- and post-translational modification. When detecting or determining the level of a protein in a sample, the ability to differentiate between different forms of a protein depends upon the nature of the difference and the method used to detect or measure the protein level. For example, an immunoassay using a monoclonal antibody will detect all forms of a protein containing the epitope to which the antibody is raised and will not distinguish between forms. However, a sandwich immunoassay that uses two antibodies directed against different epitopes on a protein will distinguish between forms of the protein that contain both epitopes and those that contain only one of the epitopes will be detected.

In a method of the invention the assay method used to determine the level of the biomarker proteins preferably detects all forms of the specific biomarker protein. Preferably at least all biologically active forms of the biomarker protein are detected. Preferably, all forms of the biomarker protein with at least 75% or more, preferably at least 80%, 85%, 90% or 95% or more, identity with the amino acid sequence of the biomarker will be detected in the method of the invention. Preferably the amino acid sequence of the biomarker is as given in FIG. 3.

Methods of measuring polypeptide/protein/nucleic acid identity are well known in the art. For example the UWGCG Package provides the BESTFIT program which can be used to calculate identity (e.g. used on its default settings) (Devereux et al (1984) Nucleic Acids Research 12, p387-395).

The PILEUP and BLAST algorithms can also be used to calculate homology or line up sequences (typically on their default settings), for example as described in Altschul S.F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/).

The method of the invention preferably allows the level of biomarker (GTPCH), either protein or mRNA, with polymorphisms common in the general population to be detected. Polymorphisms can occur that do not affect the function of a protein, or the protein encoded by a mRNA, it is intended that the method of the invention will allow these to be detected. The level of the biomarker, whether protein or mRNA, present in the sample may be determined by any suitable assay which may comprise the use of any of the group comprising, enzyme assays, immunoassays, spectrometry, mass spectrometry, Matrix Assisted Laser Desorption/Ionization Time-of-Flight (MALDI-TOF) Mass Spectrometry, microscopy, northern blot, western blot, southern blot, isoelectric focussing, SDS-PAGE, PCR, quantitative RT-PCR, gel electrophoresis, protein microarray, DNA microarray, and antibody microarray, or combinations thereof. Preferably the level of the biomarker is determined using an immunoassay. An immunoassay uses an antibody or antibodies to a specific antigen to determine the levels of the antigen. In this case, an antibody or antibodies specific to GTPCH or a GTPCH precursor protein, or a fragment of GTPCH, may be used. The immunoassay may be an enzyme linked immunoassay (ELISA), a sandwich assay, a radioimmunoassay, a Western Blot, an immunoassay using a biosensor, an immunoprecipitation assay, an agglutination assay, a turbidity assay or a nephlelometric assay.

The one or more antibodies may be synthetic, monoclonal, polyclonal, bispecific, chimeric or humanised. A chimeric antibody includes portions derived from different animals. Humanised antibodies are antibodies from non-human species having one or more complementary determining regions from the non-human species and a framework region from a human immunoglobulin molecule. Chimeric and humanised antibodies can be produced by recombinant techniques well known in the art.

The one or more antibodies may comprise a tag or a label selected from the group comprising a radioactive, a fluorescent, a chemiluminescent, a dye, an enzyme, or a histidine tag or label, or any other suitable label or tag known in the art.

The level of GTPCH in a sample may be determined by measuring the level of GTPCH protein and/or mRNA in the sample.

Preferably the reference value, to which the determined levels of the biomarker are compared, is the level of the same protein or mRNA observed in one or more subjects that do not have any detectable cancer or any clinical symptoms of cancer, and have so called “normal values” of the biomarker GTPCH.

Preferably an about two fold or more increase in GTPCH levels in a sample, compared to the level in a normal tissue sample, is diagnostic of cancer.

Alternatively, the reference value may be a previous value obtained for a specific subject. This kind of reference value may be used if the method is to be used to monitor progression of a cancer or to monitor the response of a subject to a particular treatment.

When the determined level of the biomarker is compared with a reference value, an increase or a decrease in the level of the biomarker may be indicative of the cancer status of the subject.

More specifically an increase in the level of the biomarker may be indicative, or diagnostic, of cancer.

The method of the invention may also be used to monitor cancer progression and/or to monitor the efficacy of treatments administered to a subject. This may be achieved by analysing samples taken from a subject at various time points following initial diagnosis and monitoring the changes in the levels of the biomarker and comparing these levels to normal and/or reference values.

In this case reference levels may include the initial levels of the biomarker in the subject, or the levels of the biomarker in the subject when they were last tested, or both.

Preferably the method of the invention is carried out in vitro.

The subject may be mammal, and is preferably a human, but may alternatively be a monkey, ape, cat, dog, cow, horse, rabbit or rodent.

According to another aspect of the invention there is provided a kit for use in determining the cancer status of a subject comprising at least one agent for determining the level of GTPCH mRNA or protein in a sample provided by the subject.

The agent may be an enzyme, an antibody, a nucleic acid, a protein probe, a metabolite or any other suitable composition.

The agent for determining the level of GTPCH is preferably labelled. The kit may also comprise means for detecting the label.

The kit may comprise one or more capture agents for capturing GTPCH protein or mRNA, the level of which is to be determined. The capture agent may be one or more antibodies.

The capture agent or the agent for determining the level of GTPCH may be attached to a solid support. The solid support may be a chip, a microtitre plate, a bead or a resin.

The kit may also comprise a wash solution or instructions for making a wash solution. The wash solution may, alone or in combination with the capture agent, allow efficient capture of GTPCH protein and/or mRNA on a solid support for subsequent detection by, e.g., mass spectrometry or immunoassay methods.

The kit may comprise instructions for suitable operational parameters in the form of a label or separate insert. The instructions may inform a consumer about how to collect the sample, and/or how to wash the capture agent.

The kit may comprise one or more GTPCH protein and/or mRNA samples, to be used as standard(s) for calibration and comparison. The kit may also comprise instructions to compare the level of GTPCH detected in a sample with a calibration sample or chart. The kit may also include instructions indicating what level of GTPCH is diagnostic of cancer.

According to a yet further aspect, the invention provides the use of the determination of the levels of GTPCH as a means of assessing the cancer status in an individual.

According to a further aspect the invention provides a probe set comprising two or more probes capable of detecting GTPCH. The man skilled in the art will appreciate how to design suitable probes, which may, for example, be synthetic, antibody, nucleic acid or proteinacious in nature. The probe may also carry one or more labels to facilitate its detection; the label may, for example, be a radioactive, a chemiluminescent or a florescent label, but is not limited to these examples.

According to another aspect the invention provides a method of treating cancer in a subject comprising administering to the subject an agent capable of modulating the level of active GTPCH protein in a cell.

The agent may modulate the GTPCH level by acting at the transcriptional, translation and/or post translational level. Preferably the GTPCH level is modulated to reduce the level of active GTPCH protein.

The agent may be an antisense or an interfering RNA molecule, such as a small interfering RNA molecule (siRNA), designed to inhibit or reduce expression of the GTPCH protein. The agent may increase the degradation of GTPCH mRNA and/or decrease synthesis of GTPCH mRNA.

The siRNA molecules may be selected from the group comprising the sequence of Sequence ID No: 1, 2, 3, 4, 5 and 6.

| | |
|---|---|
| Sequence ID No: 1: | CCUAAACGAUGCUAUAUUUTT |
| Sequence ID No: 2: | AAAUAUAGCAUCGUUUAGGTT |
| Sequence ID No: 3: | GCCUUAUUAAUGAUUAUCUTT |
| Sequence ID No: 4: | AGAUAAUCAUUAAUAAGGCTT |
| Sequence ID No: 5: | GUAUAGAUGGUAUAGGUAUTT |
| Sequence ID No: 6: | AUACCUAUACCAUCUAUACTT |

Alternatively, the agent may be 2,4-diamino-6-hydroxypyrimidine (DAHP), which inhibits GTPCH protein activity. DAHP competes with GTP, a substrate for GTPCH, due to the structural similarity between DAHP and GTP (Gross, S. S., and Levi, R. (1992) J Biol Chem 267(36), 25722-25729).

According to a further aspect the invention provides a method of identifying compounds for treating cancer comprising screening for one or more compounds that modulate the level of active GTPCH protein in vitro and/or in vivo.

Compounds suitable for therapeutic testing may be screened initially by identifying compounds which interact with GTPCH. By way of example, screening might include recombinantly expressing GTPCH, purifying the GTPCH, and affixing the GTPCH to a substrate. Test compounds would then be contacted with the substrate, typically in an aqueous conditions, and interactions between the test compound and the GTPCH may be measured, for example, by measuring elution rates as a function of salt concentration.

In a related embodiment, the ability of a test compound to inhibit the activity of GTPCH may be measured. One of skill in the art will recognize that the techniques used to measure the activity of GTPCH will vary depending on the assay conditions. For example, the enzymatic activity of GTPCH may be assayed provided that an appropriate substrate is available and provided that the concentration of the substrate or the appearance of the reaction product is readily measurable. The ability of potentially therapeutic test compounds to inhibit or enhance the activity of GTPCH may be determined by measuring the rates of GTPCH activity in the presence or absence of the test compounds.

Test compounds capable of modulating the activity of GTPCH may be administered to patients who are suffering from or are at risk of developing cancer. For example, the administration of a test compound which decreases the activity of GTPCH may decrease the risk of cancer in a patient if the increased activity of GTPCH is responsible, at least in part, for the onset of cancer.

The skilled man will appreciate that preferred features of any one embodiment and/or aspect of the invention may be applied to all other embodiments and/or aspects of the invention.

The present invention will be further described in more detail, by way of example only, with reference to the following Figures in which:

FIG. 1—shows the role of GTPCH in the de novo pathway of BH4 synthesis from GTP and neopterin. Abbreviations: GTPCH, GTP cyclohydrolase I; PTPS, 6-pyruvoyl tetrahydropterin synthase; SR, sepiapterin reducatase; DAHP, 2,4-diamino-6-hydroxypyrimidine;

FIG. 2—shows the nucleic acid sequence of human GTPCH mRNA;

FIG. 3—shows the amino acid sequence of active human GTPCH protein;

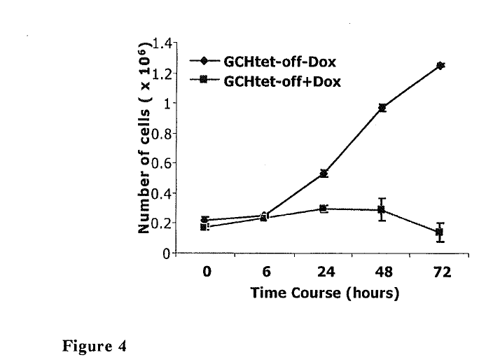

FIG. 4—shows that in vitro the over-expression of GTPCH results in cell proliferation;

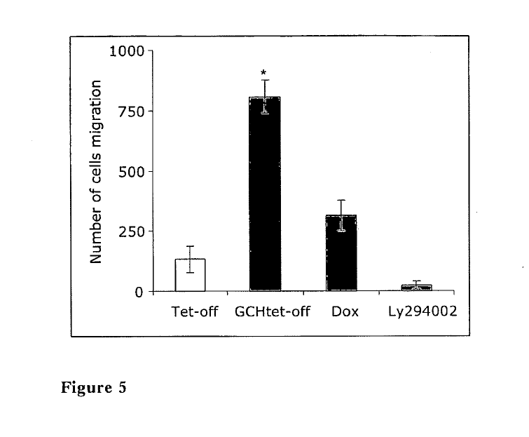

FIG. 5—shows that in vitro over-expression of GTPCH results in cell migration;

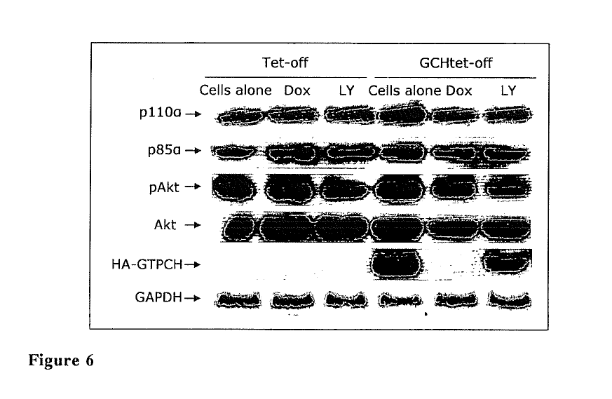

FIG. 6—shows that in vitro over-expression of GTPCH results in an increase in Akt phosphorylation in relation to levels of expression of P13K subunits;

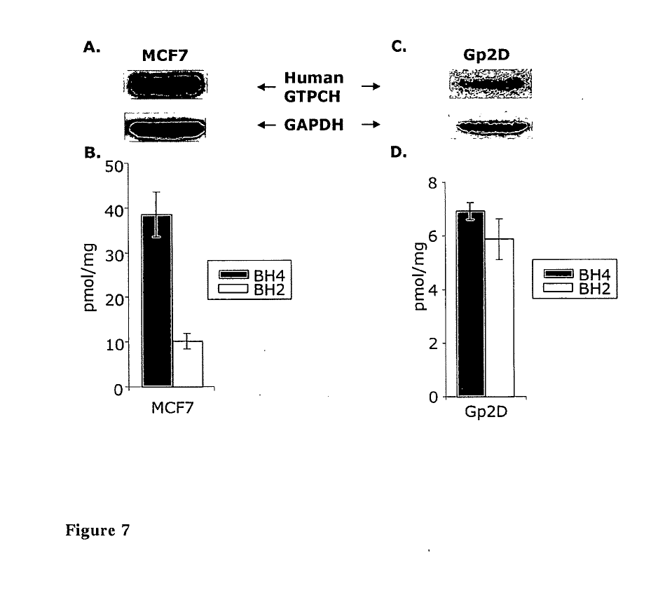

FIG. 7—shows that expression of GTPCH in cancer cell lines correlates with enzyme activity;

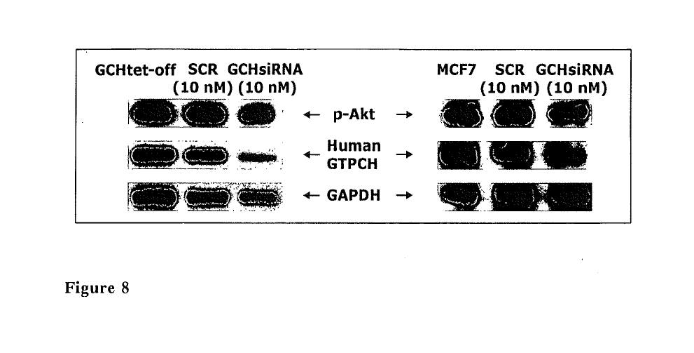

FIG. 8—shows that down regulation of GTPCH in cancer cells correlates with a reduction in Akt phosphorylation;

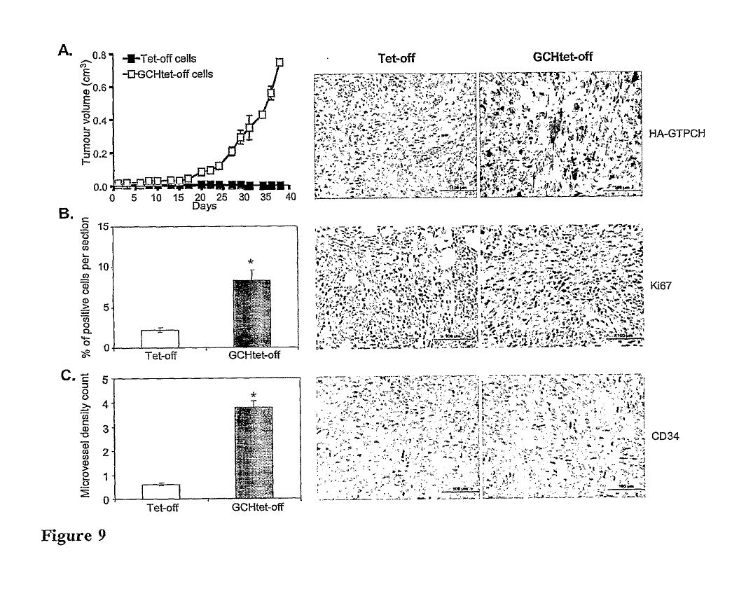

FIG. 9—shows the effect of a GCHtet-off xenograph in mice on tumour volume (FIG. 9A), appearance of Ki67 positive cells (FIG. 9B) and microvessel density count (FIG. 9C);

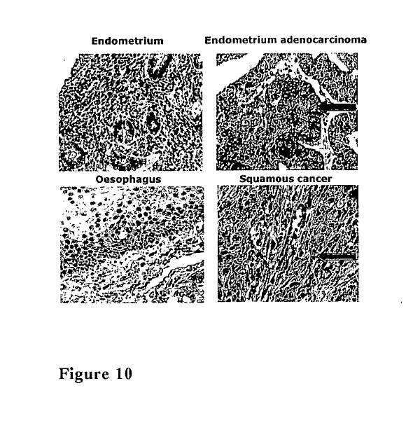

FIG. 10—shows immunohistochemical staining of samples of normal and cancerous endometrium and oesophagus tissue. The tissue is stained with anti-human GTPCH antibody. The arrows indicate the cancer cells;

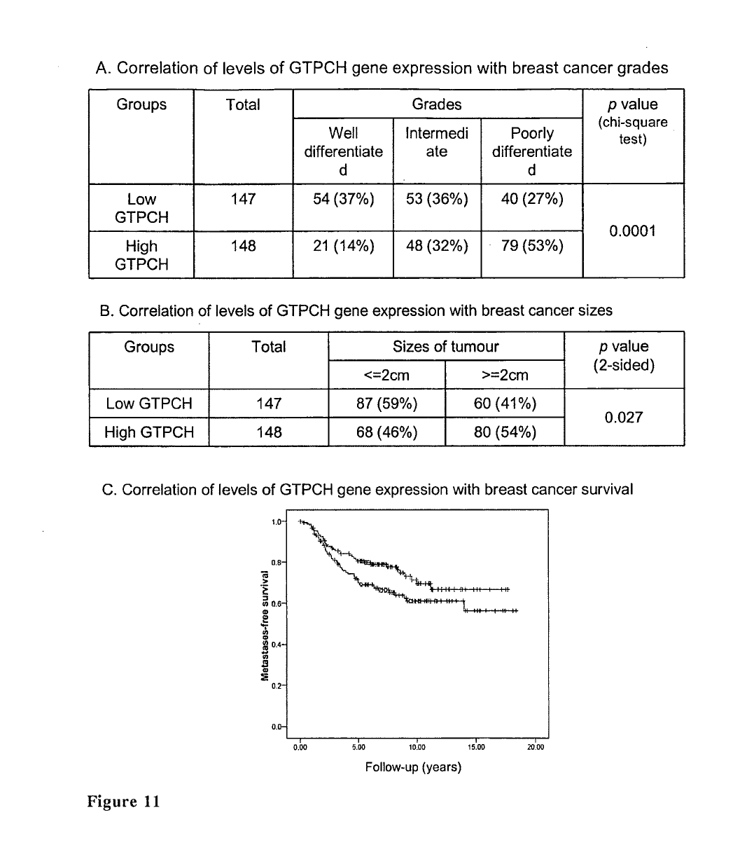

FIG. 11—shows there is a correlation between tumour grade (FIG. 11A), size (FIG. 11B) and patient survival (FIG. 11C) and GTPCH expression in breast cancer patients.

### EXAMPLE 1

### Determination of GTP Cyclohydrolase Expression in Relation to Human Cancers

In order to determine GTPCH expression in human cancers, immunohistochemical staining was undertaken on samples of human tumours held in a tissue bank. A method similar to that described by Winter et al in Cancer (2006) Vol 107 No 4 pg 757-766 was used to prepare the samples. More specifically, embedded tissue array sections were prepared and deparaffinised. The sections were then placed in 0.01M sodium citrate buffer (pH=6.0) at 100° C. for 20 minutes to retrieve the antigen epitope. The sections were then blocked in horse serum for 10 minutes and hybridised overnight with mouse anti-human GTPCH antibody (from Abnova Corporation at 1:500 dilution). The sections were then washed in PBS for 5 minutes, before being incubated with an HRP (horse radish peroxidase) anti-mouse secondary antibody (1:400 dilution) for 25 minutes, and then with substrate for 5 minutes. Finally, the sections were counter stained and mounted for the examination. The sections were scored using the H-Scoring method.

For the H-Score assessment, 10 fields were chosen at random at ×400 magnification and the staining intensity in the cell nuclei was scored as 0, 1, 2 or 3 corresponding to the presence of negative, weak, intermediate and strong brown staining, respectively. The total number of cells in each field and the number of cells stained at each intensity were counted. The average percentage positive was calculated and the following formula was applied:

IHA H-score=(% of cells stained at intensity category 1×1)+(% of cells stained at intensity category 2×2)+(% of cells stained at intensity 3×3).

An H-Score between 0 and 300 was obtained, where 300 was equal to 100% of tumour cells stained strongly (3+).

The experimental results show there was excessive GTPCH protein expression in tissue arrays from patients with various cancers, in particular lung, breast, head and neck cancers as well as lymphomas, compared with normal tissues. For example, in lung cancer a 2 to 3 fold increase in GTPCH protein levels were seen.

Generally, in normal tissues, including adrenal, intestine, skin, cervix, endometrium, ovary, pancreas, stomach, tonsil duct, testis, salivary gland, lymph node, kidney, oesophagus, trachea, thymus, lung, liver, breast, vas deferen, appendix, respiratory epithelium, bladder, placenta, parathyroid, a mean H-score for GTPCH expression of 2.87 was observed. Whereas in a variety of tumours, including adrenal tumour, phaeochromocytoma, ovary serous tumour, basal cell tumour, goitre, breast duct tumour, testis semio tumour, fibroid uterus tumour, parat aden tumour, large intestine carcinoma, adrenal carcinoma, testis teratoma, endometrium tumour, lung adno carcinoma, Warthin's tumour, mixed salivary gland tumour, liver tumour, thyroid tumour, pancreas tumour, renal tumour, oesophagus squamous tumour, HD-classic and MZL tumour, a mean H-score of 5.89 was observed, demonstrating that GTPCH levels are at least 2-fold greater in tumour tissues than in normal tissues.

Table 1 below shows the results of GTPCH staining in 328 sections of tissue array from a total 187 cases with different types of tumours.

| | A variety | Head & neck | Lung | |
|---|---|---|---|---|
| Types of tumours | of tumours | tumours | cancer | Lymphomas |
| Number of patients | 49 | 75 | 24 | 39 |
| Patients with | 34.9% | 48% | 41.7% | 58.9% |
| GTPCH positive | (17/49) | (36/75) | (10/24) | (23/39) |
| staining | | | | |

### EXAMPLE 2

### GTPCH Over-Expression in Mouse Cell Lines Promotes Cell Proliferation

Using a murine fibroblast cell line derived from 3T3 cells (GCHtet-off) in which the expression of GTPCH is regulated by doxycycline (dox) a promotion of cell proliferation was observed in vitro when GTPCH was over-expressed. The results are illustrated in FIG. 4. In GCHtet-off cells the presence of dox down-regulates GTPCH expression.

The GCHtet-off cells were constructed by transfecting 3T3 cells with a Tet-off plasmid carrying the GCH gene and a hygromycin resistance gene. Cells transfected with the plasmid that are hygromycin resistant, express human GTPCH and are capable of responding to doxycycline regulation were selected. When GCHtet-off cells were incubated with 1 μg/ml of Dox, GTPCH expression was switched off. There is no detectable level of endogenous GTPCH in normal 3T3 cells.

### EXAMPLE 3

### GTPCH Over-Expression in Mouse Cell Lines Promotes Cell Migration

2×10⁴ GCHtet-off cells (as described above) were incubated in serum free DMEM±Dox (1 μg/ml), or Ly294002 (10 μM) in a Boyden chamber with an 8 μM pore size polycarbonate membrane. Empty vector transfected Tet-off cells were used as control. (The control cells contained the same plasmid as the GCHtet-off cells but without the GTPCH gene.) After 24 hours, cells that migrate through the pores to the lower membrane were stained with Reastain Diff-Quick kit (Reagena) and counted. The results demonstrate that GTPCH over-expression facilitates the cell migration, which is 3-fold greater in GCHtet-off cells than control Tet-off cells. The results are illustrated in FIG. 5. The data is expressed as means±S.E.M (n=6, * p<0.05 GCHtet-off vs Tet-off, Dox and Ly294002).

The addition of either Dox or Ly294002 to the cells results in a significant reduction in the number of cells migrating, indicating that the migration is P13K/Atk signalling dependent. Ly294002 is an inhibitor of the P13K/Atk signalling pathway.

The in vitro migration of cells is used to model in vivo metastasis.

### EXAMPLE 4

### GTPCH Over-Expression in Mouse Cell Lines Increases Akt Phosphorylation in Relation to Levels of Expression of PI3K Subunits

GCHtet-off and control Tet-off cells were incubated±1 μg/ml of Dox for 48 hours or 10 μM of Ly294002 (LY) for one hour. The cell lysates were prepared for measuring PI3K subunits (p110α and p85α), Akt and HA-tagged GTPCH expression with Western blot. The results illustrated in FIG. 6 demonstrate that GTPCH over-expression increases Akt phosphorylation and PI3K expression, and that these levels are reduced to basal levels in the presence of Dox or LY.

### EXAMPLE 5

### GTPCH Expression in Human Cancer Cell Lines Correlates with Enzymatic Activity

Human cancer cell lines from breast (MCF7) and colon (Gp2D) were cultured in serum free medium for 48 hours and pelleted to allow expression of human GTPCH to be determined by Western blot analysis (FIG. 7A and FIG. 7C) and BH4/total biopterin levels to be determined by enzymatic assay, which represents GTPCH enzymatic activity in the cancer cells (FIG. 7B and FIG. 7D). The results demonstrate that human GTPCH expression and BH4 levels are high in MCF7 and GP2D cells.

### EXAMPLE 6

### GTPCH Expression is Downregulated by GCH1 siRNA, with a Corresponding Reduction in Akt Phosphorylation

GCHtet-off cells (described above) and MCF7 cells (4×10⁵/well) were cultured in a 24-well plate and transfected with 10 nM of GCH1 siRNA (comprising the sequences of Seq ID Nos: 1 and 2) and scramble siRNA (SCR), respectively using Lipofactamine RNAiMAX. After 48 hours, the cells were harvested and the expression of human GTPCH, phosphor-Akt and GAPDH was determined. The results are illustrated in FIG. 8. A correlation between human GTPCH expression and phosphor-Akt was observed.

In one embodiment, a possible role for GTPCH over-expression in tumorogenesis may be by activation of Akt via P13K interaction. This possibility is supported by the data presented herein.

The PI3K family is a group of enzymes that regulates several key signal transduction pathways. The three subfamilies of PI3K differ in subunit composition and substrate specificity, but only Class IA is clearly involved in oncogenesis; it comprises heterodimers of any one of the catalytic subunits (p110α, p110β, or p110γ) complexed with any one of the regulatory subunits (p85α, p85β, or p55γ). When p85-p110 complexes are activated by growth factors or oncogenes, they convert phosphatidylinositol (4,5)-bisphosphate (PIP2) to phosphatidylinositol (3,4,5)-bisphosphate (PIP3) in the plasma membrane. As a result of this translocation, PIP3 acts as a second messenger and then activates downstream pathways, Akt signalling in particular.

The kinase Akt is a central player in signal transduction protecting cells from apoptosis, promoting cell proliferation and growth. To interact with PIP3, Akt is recruited from the cytosol to the plasma membrane and then phosphorylated. Its two key regulatory sites, threonine-308 and serine-473, are frequently modified, so activating a wide range of downstream tumourigenic signals. For example, constitutively active Akt directly phosphorylates BAD and caspase-9 to inhibit cell apoptosis, glycogen synthase kinase-3β (GSK-3β) and mammalian target of rampamycin (mTOR) to enhance cell proliferation and growth, respectively, the key pathological characteristics of human cancers. Essentially, the p85α subunit of PI3K is partially responsible for GTPCH expression/enzymatic activity. In line with these findings, it is hypothesised that one of the crucial contributions of GTPCH in tumourigenesis is activation of Akt via PI3K interaction.

### EXAMPLE 7

### In Vivo Assessment of GTPCH Expression in Tumour Progression

Using Balb/c SCID mice xenotransplanted with GCHtet-off cells, the effect of GTPCH over-expression on neoplastic transformation in vivo was observed. As can be seen from the results presented in FIGS. 9A, 9B and 9C. From FIG. 9A it can be seen that when GTPCH is over-expressed the tumour volume increases dramatically. FIG. 9B illustrates that GTPCH over-expression leads to a 5-fold increases in Ki67 positive cells. Wherein Ki67 is a positive marker of cell proliferation. FIG. 9C illustrates that the microvessel density count increases with GTPCH over-expression.

Mice were xenotranplantated with GCHtet-off by subcutaneously injecting 1×10⁷ cells into female mice. 3T3tet-off, the empty vector only transfected cells, were used as a control

### EXAMPLE 8

### GTPCH Over-Expression Observed in Tumour Sections

Immunohistochemical staining of tissue sections and tissue arrays from a variety of cancer samples demonstrated that a number of cancers display increased levels of GTPCH expression. In particular, FIG. 10 compares normal and cancerous endometrium and oesophagus tissue stained with an anti-human GTPCH antibody, demonstrating that GTPCH levels are clearly enhanced in the cancer tissue. Similar results were also obtained with breast cancer tissue.

### EXAMPLE 9

### Assessment of GTPCH Expression in a Clinical Setting and Correlation to Therapy and Prognosis in Patients with Breast Cancer

Analysis of a set of published data (from Chang H Y et al. Proc Natl Acad Sci USA 2005; 102(10):3738-43) revealed that high GTPCH RNA levels were significantly correlated with high grade, large, node positive tumours in 295 breast cancer patients.

As illustrated in FIG. 11, a high level of GTPCH expression was significantly correlated with poorly differentiated breast cancer grade (FIG. 11A) and large tumour size (FIG. 11B), although there was only a borderline difference between survival after nearly 20 years follow-up (FIG. 11C; upper line—low GTPCH and lower line—high GTPCH).

1-29. (canceled)

30. A method of determining, the cancer status in a subject comprising the determination of the levels of GTPCH in the subject.

31. The method of claim 30, further comprising the steps of:
(a) providing a sample of material obtained from a subject;
(b) determining the level GTPCH in the sample; and
(c) comparing the level determined in (b) with one or more reference values.

32. The method of claim 30 which is used in conjunction with an assessment of clinical symptoms.

33. The method of claim 30 wherein the method is for use in one or more of the following: the diagnosis of cancer in a subject; the assessment of the chance of a subject developing cancer; advising on the prognosis for a subject with cancer; monitoring disease progression; and monitoring the effectiveness or response of a subject to a treatment.

34. The method of claim 30 wherein the cancer is selected from the group comprising cancer of the lung, breast, brain, breast, pancreas, neck, prostate, bone, ovary, colon, liver, testis, endometrium, muscle, fat tissue, fibrous tissue, soft tissue, squamous tissue, skin tissue and lymphoma tissue, or combinations thereof.

35. The method of claim 30 wherein the sample material obtained from the subject comprises one or more of whole blood, blood serum, blood plasma, urine, mucous, fat tissue (adipose), lung tissue, brain tissue, pancreatic tissue, neck tissue, testis tissue, endometrium tissue, liver tissue, bone tissue, gastro-intestinal tissue, colon tissue, breast tissue, prostate tissue, lymphoma tissue, muscle tissue, fibrous tissue, soft tissue, squamous tissue, skin tissue, ovary tissue, fat tissue, lymphoma tissue or any other tissue or bodily fluid.

36. The method of claim 30 wherein the wherein the determination of the level of GTPCH in a sample comprises the detection of a polypeptide or an mRNA with at least 65% sequence identity to the sequence of GTPCH in FIG. 2 or 3 respectively.

37. The method of claim 30 wherein the reference value is the level of GTPCH protein or mRNA observed in one or more subjects that do not have any detectable cancer.

38. The method of claim 30 wherein the reference value is a previous value obtained for a specific subject.

39. The method of claim 30 wherein a level of GTPCH determined in (b) greater than the reference value is diagnostic that the sample is from a cancer.

40. The method of claim 39 wherein the level of GTPCH determined in (b) must be at least twice the reference value to be diagnostic of cancer.

41. The method of claim 30 wherein the method is carried out in vitro.

42. The method of claim 30 wherein the subject is a mammal.

43. A method of treating cancer in a subject comprising administering to the subject an agent capable of modulating the level of active GTPCH protein in a cell.

44. The method of claim 43 wherein the agent modulates the GTPCH level by acting at the transcriptional, translation and/or post translational level.

45. The method of claim 43 wherein the agent is an antisense or an interfering RNA molecule, designed to inhibit or reduce expression of the GTPCH protein.

46. The method of claim 45 wherein the siRNA molecules is selected from the group comprising the sequence of Sequence ID No: 1, 2, 3, 4, 5 and 6.

47. The method of claim 43 wherein the agent is 2,4-diamino-6-hydroxypyrimidine (DAHP).

48. A method of identifying compounds for treating cancer comprising screening for one or more compounds that modulate the level of active GTPCH protein in vitro and/or in vivo.
